(19) United States
(12) Patent Application Publication
(10) Pub. No.: US 2017/0119313 A1
(43) Pub. Date: May 4, 2017

(54) Wellness Monitoring Method and System with Temperature-Based Forehead Contact Detection

(71) Applicant: Sharp Laboratories of America, Inc., Camas, WA (US)

(72) Inventors: Richard Eric Helvick, Portland, OR (US); Geoff Harvey, Gamas, WA (US)

(73) Assignee: Sharp Laboratories of America, Inc., Camas, WA (US)

(21) Appl. No.: 14/925,868

(22) Filed: Oct. 28, 2015

(51) Int. Cl. A61B 5/00; A61B 5/0205

(52) U.S. Cl. CPC A61B 5/6844; A61B 5/02055; A61B 5/681; A61B 5/6898; A61B 5/021

(57) ABSTRACT

A wellness monitoring method and system with temperature-based forehead contact detection comprises a sensor device configured for placement in contact with a user's forehead and a mobile computing device wirelessly coupled with the sensor device. The system uses temperature readings taken by the sensor device to detect when contact between the sensor device and the user's forehead is established and lost and, as a result, when to start and stop wellness measurements.

### BACKGROUND OF THE INVENTION

The present invention relates to physiological monitoring and, more particularly, monitoring human wellness using a sensor device placed in contact with the forehead.

Wellness monitoring systems that take physiological readings at the forehead of a human subject and output wellness information based on those readings on a smart phone are known. These systems typically include a sensor device for placement in contact with a system user's forehead and a smart phone running a wellness application. The sensor device and smart phone conduct wellness measurement sessions over a wireless link in order to generate and output the wellness information.

Such wellness monitoring systems perform suboptimally if the physiological readings are taken when the sensor device is not in proper contact with the user's forehead. If the physiological readings are started before contact is established with the user's forehead or continue after contact is lost, the wellness information outputted on the smart phone will be error-prone and the sensor device's battery will be wasted.

One way to synchronize the physiological reading period to the contact period between the sensor device and the user's forehead is explicit user input. For example, the sensor device or the smart phone can be configured with an on/off (or start/stop) button which the user presses when the sensor device is placed on and removed from the user's forehead. However, requiring the user to explicitly indicate the start and end of forehead contact places an extra burden on the user and can introduce measurement error since the user may not be able to reliably determine when the sensor device is in proper contact with the forehead.

### SUMMARY OF THE INVENTION

The present invention provides a wellness monitoring method and system with temperature-based forehead contact detection. The system comprises a sensor device configured for placement in contact with a user's forehead and a mobile computing device wirelessly coupled with the sensor device. The system uses temperature readings taken by the sensor device to detect when contact between the sensor device and the user's forehead is established and lost and, as a result, when to start and stop wellness measurements. By using temperature readings taken by the sensor device to determine when to start and the stop wellness measurements, the reliability of system output and system longevity are improved without imposing extra burdens on the user.

In one aspect of the invention, a wellness monitoring system comprises a sensor device configured to take measurements of a plurality of physiological parameters; and a mobile computing device wirelessly coupled with the sensor device configured to output wellness information determined from the measurements, wherein the system is configured to start the measurements when an in-contact state is detected between the sensor device and a forehead, stop the measurements when a non-contact state is detected between the sensor device and the forehead, and transition between the in-contact state and the non-contact state based on one or more temperature readings taken by the sensor device.

In some embodiments, the system is configured to transition from the non-contact state to the in-contact state in response to a determination that consecutive ones of the temperature readings are above a rising temperature threshold.

In some embodiments, the rising temperature threshold is between 92 and 96 degrees Fahrenheit, inclusive.

In some embodiments, the system is configured to transition from the in-contact state to the non-contact state in response to a determination that consecutive ones of the temperature readings are below a falling temperature threshold.

In some embodiments, the falling temperature threshold is between 87 and 91 degrees Fahrenheit, inclusive.

In some embodiments, the system is configured, upon transitioning from the non-contact state to the in-contact state, to initiate a wellness measurement session.

In some embodiments, the system is configured, upon transitioning from the non-contact state to the in-contact state, to resume a suspended wellness measurement session.

In some embodiments, the system is configured, upon transitioning from the non-contact state to the in-contact state, to output on the mobile computing device an indication that a wellness measurement session is in progress.

In some embodiments, the system is configured, upon transitioning from the in-contact state to the non-contact state, to terminate a wellness measurement session.

In some embodiments, the system is configured, upon transitioning from the in-contact state to the non-contact state, to suspend a wellness measurement session that may be resumed.

In some embodiments, the system is configured, upon transitioning from the in-contact state to the non-contact state, to output on the mobile computing device an indication to reestablish contact between the sensor device and the forehead.

In some embodiments, the wellness information comprises heart information.

In some embodiments, the wellness information comprises blood oxygen information.

In some embodiments, the wellness information comprises blood pressure information.

In some embodiments, the wellness information comprises stress information.

In some embodiments, the mobile computing device is a smart phone.

In some embodiments, the mobile computing device is a smart watch.

In another aspect of the invention, in a wellness monitoring system having a sensor device configured to take measurements of a plurality of physiological parameters and a mobile computing device wirelessly coupled with the sensor device configured to output wellness information determined from the measurements, a wellness monitoring method comprises starting by the system the measurements when an in-contact state is detected between the sensor device and a forehead; stopping by the system the measurements when a non-contact state is detected between the sensor device and the forehead; and transitioning by the system between the in-contact state and the non-contact state based on one or more temperature readings taken by the sensor device.

In some embodiments, the transitioning step comprises transitioning from the non-contact state to the in-contact state in response to a determination that consecutive ones of the temperature readings are above a rising temperature threshold.

In some embodiments, the transitioning step comprises transitioning from the in-contact state to the non-contact state in response to a determination that consecutive ones of the temperature readings are below a falling temperature threshold.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a wellness monitoring system in embodiments of the invention.

FIG. 2 shows the relationship between the contact state of the system's sensor device and the measuring state of the system.

FIG. 3 is a perspective view of the sensor device.

FIG. 4 is a component view of the sensor device.

FIG. 5 is a component view of the system's mobile computing device.

FIG. 6 shows a method by which the system transitions from the non-contact state to the in-contact state.

FIG. 7 shows a method by which the system transitions from the in-contact state to the non-contact state.

FIG. 8 shows a message outputted on the mobile computing device when in the non-contact state.

FIG. 9 shows a message outputted on the mobile computing device when in the in-contact state.

### DETAILED DESCRIPTION OF A PREFERRED EMBODIMENT

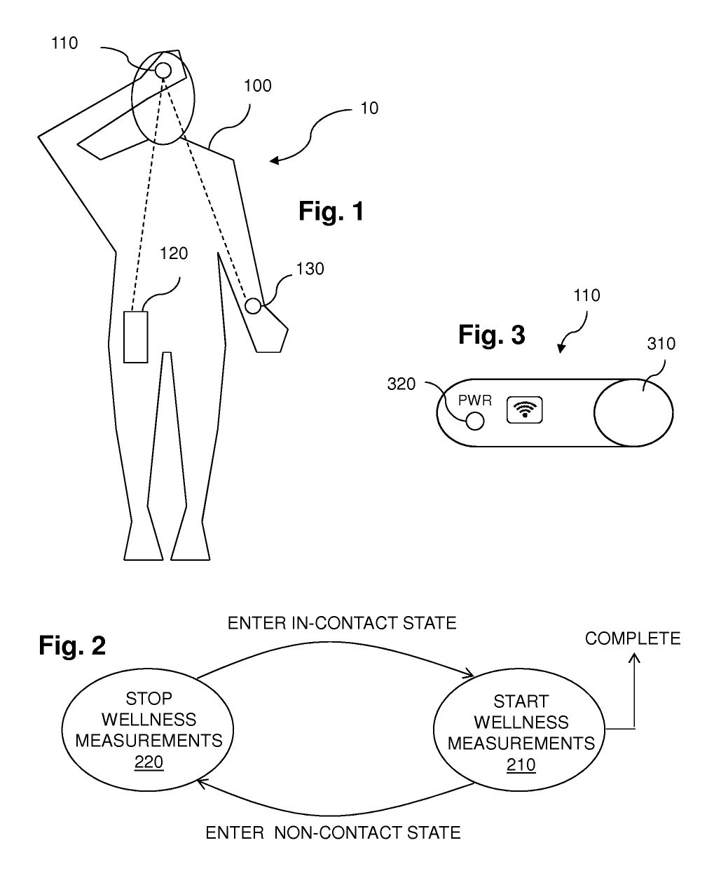

FIG. 1 shows a wellness monitoring system 10 in embodiments of the invention. System 10 includes a sensor device 110 configured for placement in contact with the forehead of a user 100 for the purpose of measuring physiological parameters of user 100, such as body temperature, heart rate, blood oxygen saturation and blood pressure. Sensor device 110 communicates over wireless links with mobile computing devices, such as a smart phone 120 and smart watch 130, to convey the measurements. The measurements are consumed by a wellness application running on the mobile computing devices to determine and output wellness information for user 100. Wellness information may include the measurements themselves, such as body temperature, heart rate, blood oxygen saturation and blood pressure, and information derived from the measurements such as a stress indicator or overall wellness score. Wireless communication between sensor device 110 and the mobile computing devices is realized using a short-range wireless communication protocol, such as Bluetooth, Infrared Data Association (IrDA) or ZigBee. The mobile computing devices each have a video display for outputting the wellness information as well as status messages to user 100. In some embodiments, system 10 has a single mobile computing device.

System 10 provides wellness information to user 100 by conducting wellness measurement sessions. In a wellness measurement session, sensor device 110 measures a plurality of physiological parameters at the forehead of user 100 over a measurement period, and mobile computing devices, such as smart phone 120 and smart watch 130, output wellness information determined from the measurements. In some embodiments, the measurements taken by sensor device 110 are transmitted over the wireless links and resolved to wellness information by a wellness application running on the mobile computing devices. In other embodiments, the measurements taken by sensor device 110 are resolved to wellness information on the sensor device 110 and the wellness information is transmitted to the mobile computing devices over the wireless links.

System 10 starts a wellness measurement session upon detecting an in-contact state between sensor device 110 and the forehead of user 100. System 10 stops the session upon detecting a non-contact state between sensor device 110 and the forehead of user 100 or when all session tasks have been successfully completed, whichever occurs first. If the non-contact state is detected while the session is in-progress, the session is stopped immediately regardless of how far or how long the session has progressed. In some embodiments, when the session is stopped, the session is merely suspended and is resumed if the in-contact state is re-detected within a predetermined time period. In other embodiments, when the session is stopped, the session is terminated immediately with no opportunity for resumption.

The relationship between the contact state of sensor device 110 and the measuring state of system 10 is shown in FIG. 2. When an in-contact state is detected between sensor device 110 and the forehead of user 100, the wellness measurement session starts 210. If a non-contact state between sensor device 110 and the forehead of user 100 is thereafter detected before all session tasks have been successfully completed, the session stops 220.

Importantly, sensor device 110 measures temperature periodically even when system 10 is in the non-contact state. In some embodiments, this period is 0.5 seconds. These temperature measurements taken outside of a wellness measurement session are used to detect entry (or re-entry) into the in-contact state and, as a result, when to start (or resume) a wellness measurement session.

FIG. 3 shows sensor device 110 in some embodiments. Sensor device 110 in these embodiments is tube-shaped. Sensor device 110 has a substantially flat forehead contact surface 310 for placement in abutment with the forehead of user 100 and a cylindrical handling surface having a power button 320. When user 100 wishes to initiate a wellness measurement session, user 100 presses power button 320 to power-on sensor device 110 and places forehead contact surface 310 against the forehead. The wellness measurement session begins once an in-contact state between sensor device 110 and the forehead of user 100 is detected. User 100 may then allow the session to proceed to successful completion by continuing to hold sensor device 110 against the forehead or terminate the session prior to completion by removing sensor device 110 from the forehead for a sustained period or pressing power button 320 to power-down sensor device 110. In some embodiments, sensor device 110 is battery-powered. Naturally, sensor device 110 may come in other shapes and various sizes.

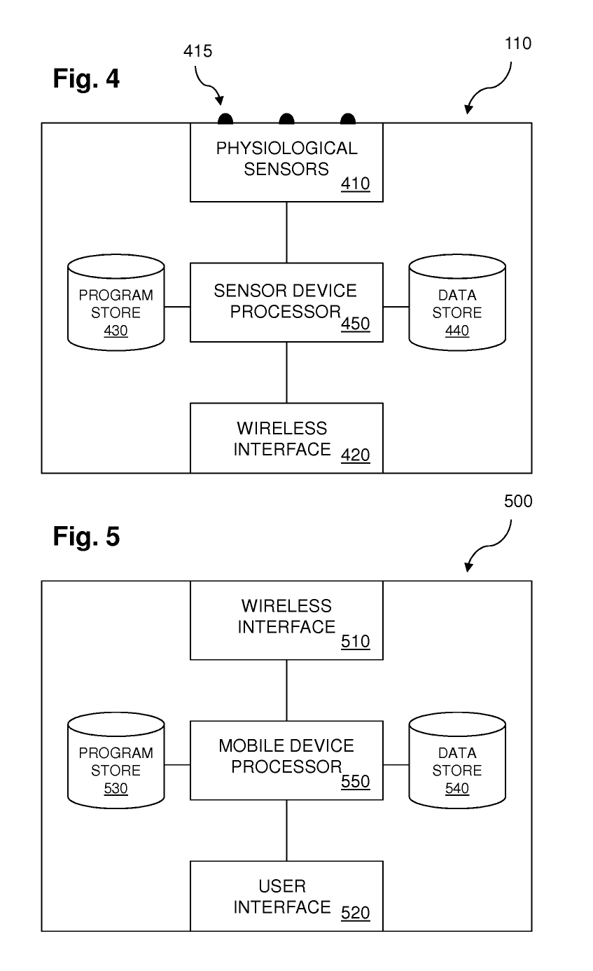

FIG. 4 shows components of sensor device 110 in some embodiments. The components include a plurality of physiological sensors 410, a wireless interface 420, a program store 430 and a data store 440, all of which are communicatively coupled with a sensor device processor 450.

Physiological sensors 410 include a multiple of sensors, which may include electrical and biochemical sensors, and which measure different physiological parameters of user 100 at the forehead. One of physiological sensors 410 is a body temperature sensor 415. Other physiological sensors 410 may include, by way of example, a heart sensor, a blood oxygen sensor and a blood pressure sensor. Under the control of sensor device processor 450, physiological sensors 410 measure their respective physiological parameters. When a wellness measurement session is in-progress, all of physiological sensors 410 take measurements. When a session is not in-progress, only body temperature sensor 415 takes measurements. The readings taken by physiological sensors 410 are processed and stored in data store 440 under the control of sensor device processor 450 and are transmitted to at least one mobile computing device, such as smart phone 120 or smart watch 130, via wireless interface 420.

Wireless interface 420 is a bidirectional wireless data communication interface, such as a Bluetooth, IrDA or ZigBee interface. Under the control of sensor device processor 450, wireless interface 420 transmits the readings taken by physiological sensors 410 to at least one mobile computing device.

Sensor device processor 450 is a microprocessor which executes program instructions from program store 430 to control physiological sensors 410 to take readings, stop taking readings, process readings, store and retrieve readings to and from data store 440, and transmit readings to at least one mobile computing device in wellness measurement sessions. Sensor device processor 450 exercises these controls in conformance with instructions from the mobile computing device received via wireless interface 420. Body temperature sensor 415 takes periodic temperature readings which are processed, stored, retrieved and transmitted under the control of sensor device processor 450 even when no wellness measurement session is in-progress.

FIG. 5 shows components of a mobile computing device 500, such as smart phone 120 or smart watch 130, in some embodiments. The components include a wireless interface 510, a user interface 520, a program store 530 and a data store 540, all of which are communicatively coupled with a mobile device processor 550.

Wireless interface 510 is a bidirectional wireless data communication interface, such as a Bluetooth, IrDA or ZigBee interface. Wireless interface 510 receives the physiological readings, including temperature readings, from sensor device 110.

User interface 520 is an input/output component for receiving input from user 100 and transmitting output to user 100. User interface 520 includes a video display whose display content is driven by mobile device processor 550.

Mobile device processor 550 is a microprocessor which executes program instructions of a wellness application installed in program store 530. Mobile device processor 550 receives the readings from sensor device 110 via wireless interface 510, processes the readings, and stores the readings in data store 540. Mobile device processor 550 uses the readings taken by all of physiological sensors 410 to programmatically determine wellness information to be outputted to user 100 via user interface 520 at the conclusion of successfully completed wellness measurement sessions. Mobile device processor 550 additionally uses temperature readings taken by body temperature sensor 415 to detect when contact between sensor device 110 and the forehead of user 100 is established and lost and, as a result, when to start and stop wellness measurement sessions. Mobile device processor 550 generates wellness measurement session status messages to be outputted to user 100 via user interface 520 and wellness measurement session instructions to be transmitted to sensor device 110 via wireless interface 510 based on the detected contact state of sensor device 110 with the forehead of user 100.

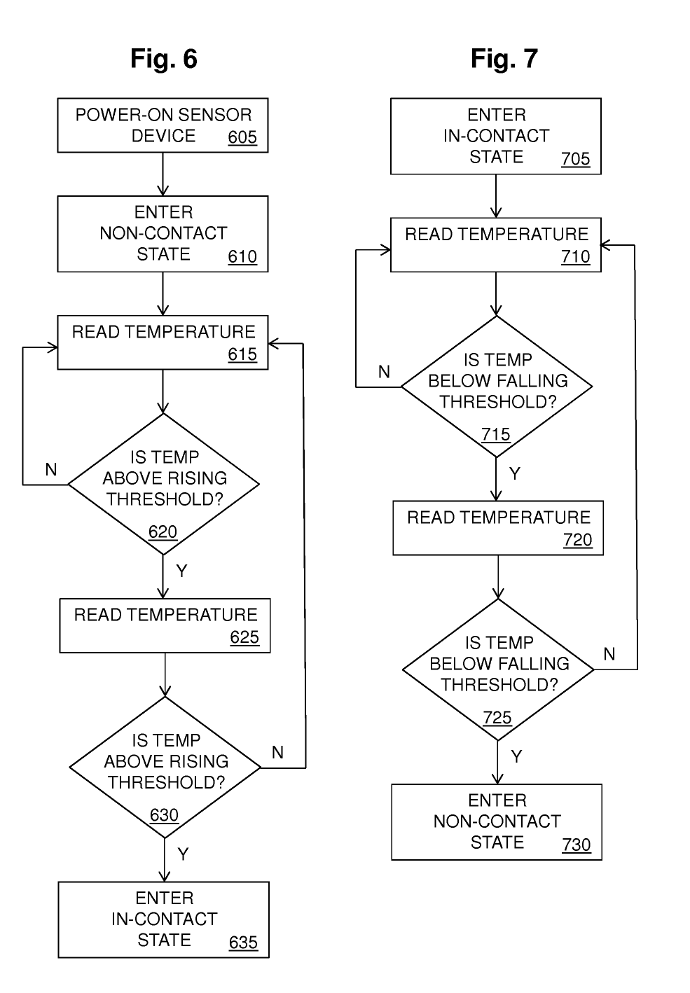

FIG. 6 shows a method by which wellness monitoring system 10 transitions from the non-contact state to the in-contact state based on temperature readings. At the outset, user 100 powers-on sensor device 110 (605). Sensor device 110 initializes to the non-contact state (610). In the non-contact state, body temperature sensor 415 takes periodic temperature readings while other physiological sensors 410 are inactive.

Body temperature sensor 415 takes a first temperature reading (615) and the reading is sent to mobile computing device 500 (e.g., smart phone 120 or smart watch 130). The wellness application running on mobile computing device 500 determines whether the reading is above a predetermined rising temperature threshold (620). In some embodiments, the rising temperature threshold is preconfigured to a setting between 92 and 96 degrees Fahrenheit, inclusive, such as to 94° F. The rising temperature threshold is preferably set below normal body temperature to account for human body temperature variation, hysteresis in the temperature sensor readings and regional temperature disparities at the forehead. With regard to the last noted reason, the forehead has warmer and cooler regions and the in-contact state should be detected when sensor device 110 is in contact with one of the cooler regions.

If the first temperature reading is below the rising temperature threshold, contact between sensor device 110 and the forehead of user 100 is presumed to not exist. System 10 remains in the non-contact state and the flow returns to Step 615.

On the other hand, if the first temperature reading is above the rising temperature threshold, an attempt is made to confirm contact between sensor device 110 and the forehead of user 100 using the next temperature reading. Accordingly, body temperature sensor 415 takes a second temperature reading (625) and the reading is sent to mobile computing device 500 where the wellness application determines whether it is above the predetermined rising temperature threshold (630).

If the second temperature reading is below the rising temperature threshold, contact between sensor device 110 and the forehead of user 100 is not confirmed. System 10 remains in the non-contact state and the flow returns to Step 615.

On the other hand, if the second temperature reading is above the rising temperature threshold, contact between sensor device 110 and the forehead of user 100 is confirmed and system 10 transitions to the in-contact state (635).

FIG. 7 shows a method by which system 10 transitions from the in-contact state to the non-contact state based on temperature readings. This method begins with system 10 in the in-contact state (705) where all of physiological sensors 410, including body temperature sensor 415, take readings.

Body temperature sensor 415 takes a first body temperature reading (710) and the reading is sent to mobile computing device 500. The wellness application running on mobile computing device 500 determines whether the reading is below a predetermined falling temperature threshold (715). In some embodiments, the falling temperature threshold is preconfigured to a setting between 87 and 91 degrees Fahrenheit, inclusive, such as to 89° F. The falling temperature threshold is preferably set well below normal body temperature to account for human body temperature variation, regional temperature disparities at the forehead, and to avoid stopping an in-progress wellness measurement session in the event sensor device 110 loses contact with the forehead of user 100 for a brief time.

If the first temperature reading is above the falling temperature threshold, contact between sensor device 110 and the forehead of user 100 is presumed to exist. System 10 remains in the forehead contact state and the flow returns to Step 710.

On the other hand, if the first temperature reading is below the falling temperature threshold, an attempt is made to confirm loss of contact between sensor device 110 and the forehead of user 100 in the next reading. Accordingly, temperature sensor 415 takes a second temperature reading (720) and the reading is sent to mobile computing device 500 where the wellness application determines whether the reading is below the predetermined falling temperature threshold (725).

If the second temperature reading is above the falling temperature threshold, loss of contact between sensor device 110 and the forehead of user 100 is not confirmed. System 10 remains in the forehead contact state and the flow returns to Step 710.

On the other hand, if the second temperature reading is below the falling temperature threshold, loss of contact between sensor device 110 and the forehead of user 100 is confirmed and system 10 transitions to the non-contact state (730). In some embodiments, upon entering the non-contact state, the current wellness measurement session is merely suspended and is resumed if the in-contact state is reestablished within a predetermined time. In these embodiments, physiological readings taken in the session prior to the suspension and the session state are saved in data store 440 and data store 540. In other embodiments, upon entering the non-contact state, the session is immediately terminated with no opportunity for resumption and physiological readings taken in the session prior to suspension of the session and session state are not saved.

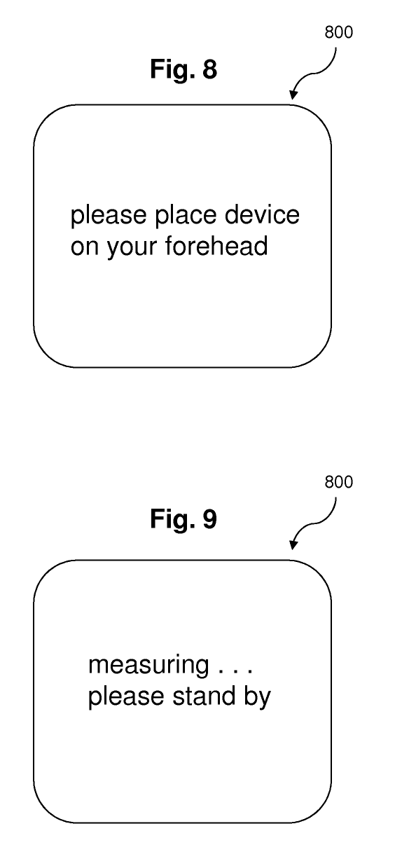

In the non-contact state, system 10 operates as follows in some embodiments. On sensor device 110, body temperature sensor 415 takes periodic temperature readings while other physiological sensors 410 do not take readings. Sensor device processor 450 transmits the temperature readings to mobile computing device 500 via wireless interface 420. On mobile computing device 500, mobile device processor 550 continually performs the method of FIG. 6 using the temperature readings to determine whether to transition to the in-contact state. Mobile device processor 550 causes the wellness measurement session status message of FIG. 8, which prompts user 100 to place sensor device 110 on the forehead, to be displayed on a display screen 800 of user interface 520.

In the in-contact state, system 10 operates as follows in some embodiments. On sensor device 110, all of physiological sensors 410 take readings of their respective physiological parameters. Sensor device processor 450 transmits the readings to mobile computing device 500 via wireless interface 420. On mobile computing device 500, mobile device processor 550 continually performs the method of FIG. 7 using the temperature readings to determine whether to transition to the non-contact state. Mobile device processor 550 causes the wellness measurement session status message of FIG. 9, which informs user 100 that a wellness measurement session is in-progress, to be displayed on display screen 800. If the session is successfully completed before system 10 transitions to the non-contact state, mobile device processor 550 replaces the wellness session status message of FIG. 9 with wellness information output determined for user 100 from the readings, such as body temperature, heart rate, blood oxygen saturation, blood pressure, stress indicator and an overall wellness score.

In some embodiments, the methods of FIGS. 6 and 7 are performed by sensor device 110 rather than mobile computing device 500. In these embodiments, sensor device processor 450 uses temperature readings taken by body temperature sensor 415 to determine transitions between the in-contact state and the non-contact state. In the non-contact state, sensor device processor 450 controls body temperature sensor 415 to take periodic readings, controls other physiological sensors 410 not to take readings and provides no readings to mobile computing device 500. In the in-contact state, sensor device processor 450 controls all of physiological sensors 410 to take readings and provides these readings to mobile computing device 500. While mobile computing device 500 is receiving readings from sensor device processor 450, mobile device processor 550 causes the session status message of FIG. 9 to be displayed on display screen 800. Prior to receiving readings from sensor device processor 450 and if mobile computing device 500 stops receiving such readings before all session tasks are successfully completed, mobile device processor 550 causes the session status message of FIG. 8 be displayed on display screen 800.

It will be appreciated by those of ordinary skill in the art that the invention can be embodied in other specific forms without departing from the spirit or essential character hereof. For example, in some embodiments a single temperature reading above a rising threshold or below a falling threshold triggers a contact state transition. The present description is considered in all respects to be illustrative and not restrictive. The scope of the invention is indicated by the appended claims, and all changes that come within the meaning and range of equivalents thereof are intended to be embraced therein.

What is claimed is:

1. A wellness monitoring system, comprising:
a sensor device configured to take measurements of a plurality of physiological parameters; and
a mobile computing device wirelessly coupled with the sensor device configured to output wellness information determined from the measurements, wherein the system is configured to start the measurements when an in-contact state is detected between the sensor device and a forehead, stop the measurements when a non-contact state is detected between the sensor device and the forehead, and transition between the in-contact state and the non-contact state based on one or more temperature readings taken by the sensor device.

2. The system of claim 1, wherein the system is configured to transition from the non-contact state to the in-contact state in response to a determination that consecutive ones of the temperature readings are above a rising temperature threshold.

3. The system of claim 2, wherein the rising temperature threshold is between 92 and 96 degrees Fahrenheit, inclusive.

4. The system of claim 1, wherein the system is configured to transition from the in-contact state to the non-contact state in response to a determination that consecutive ones of the temperature readings are below a falling temperature threshold.

5. The system of claim 4, wherein the falling temperature threshold is between 87 and 91 degrees Fahrenheit, inclusive.

6. The system of claim 1, wherein the system is configured, upon transitioning from the non-contact state to the in-contact state, to initiate a wellness measurement session.

7. The system of claim 1, wherein the system is configured, upon transitioning from the non-contact state to the in-contact state, to resume a suspended wellness measurement session.

8. The system of claim 1, wherein the system is configured, upon transitioning from the non-contact state to the in-contact state, to output on the mobile computing device an indication that a wellness measurement session is in progress.

9. The system of claim 1, wherein the system is configured, upon transitioning from the in-contact state to the non-contact state, to terminate a wellness measurement session.

10. The system of claim 1, wherein the system is configured, upon transitioning from the in-contact state to the non-contact state, to suspend a wellness measurement session that may be resumed.

11. The system of claim 1, wherein the system is configured, upon transitioning from the in-contact state to the non-contact state, to output on the mobile computing device an indication to reestablish contact between the sensor device and the forehead.

12. The system of claim 1, wherein the wellness information comprises heart information.

13. The system of claim 1, wherein the wellness information comprises blood oxygen information.

14. The system of claim 1, wherein the wellness information comprises blood pressure information.

15. The system of claim 1, wherein the wellness information comprises stress information.

16. The system of claim 1, wherein the mobile computing device is a smart phone.

17. The system of claim 1, wherein the mobile computing device is a smart watch.

18. In a wellness monitoring system having a sensor device configured to take measurements of a plurality of physiological parameters and a mobile computing device wirelessly coupled with the sensor device configured to output wellness information determined from the measurements, a wellness monitoring method, comprising:
starting by the system the measurements when an in-contact state is detected between the sensor device and a forehead;
stopping by the system the measurements when a non-contact state is detected between the sensor device and the forehead; and
transitioning by the system between the in-contact state and the non-contact state based on one or more temperature readings taken by the sensor device.

19. The method of claim 18, wherein the transitioning step comprises transitioning from the non-contact state to the in-contact state in response to a determination that consecutive ones of the temperature readings are above a rising temperature threshold.

20. The method of claim 19, wherein the transitioning step comprises transitioning from the in-contact state to the non-contact state in response to a determination that consecutive ones of the temperature readings are below a falling temperature threshold.
